(19) United States
(12) Patent Application Publication
(10) Pub. No.: US 2010/0029583 A1
(43) Pub. Date: Feb. 4, 2010

(54) PROCESS FOR OBTAINING AN ACTIVE INGREDIENT WITH AN IMMEDIATE TENSOR EFFECT ON THE SKIN, ACTIVE INGREDIENT AND COMPOSITIONS

(75) Inventor: Jean Paufique, Objat (FR)

Correspondence Address: YOUNG & THOMPSON, 209 Madison Street, Suite 500, Alexandria, VA, 22314, US

(73) Assignee: SOCIETE INDUSTRIELLE LIMOUSINE D'APPLICATION BIOLOGIQUE, DITE SILAB, Objat (FR)

(21) Appl. No.: 12/442,441

(22) PCT Filed: Sep. 21, 2007
(86) PCT No.: PCT/FR07/51992

§ 371 Date: Mar. 23, 2009

(30) Foreign Application Priority Data
Sep. 22, 2006 (FR) 0653903
Mar. 8, 2007 (FR) 0753714

(51) Int. Cl. A61K 8/73; A61Q 19/08

(52) U.S. Cl. 514/54

(57) ABSTRACT

The invention relates to a method for obtaining an active ingredient having an immediate anti-wrinkle and skin-tensioning effect, characterised in that it comprises extracting and purifying high molecular weight polysaccharides from oat bran and/or fibres and/or grains, and solutioning and stabilising the polysaccharides in water. The invention also relates to the product thus obtained, to uses thereof, and to cosmetic compositions containing this active ingredient.

This invention relates to a process for obtaining a polysaccharide-rich active ingredient of high molecular weight that is derived from oat fibers and/or bran and/or seeds, having an immediate anti-wrinkle effect and/or immediate tensor effect on the skin.

The invention also relates to the active ingredient that can be obtained by this process, its uses, and the related cosmetic compositions.

To appear younger, many people want to tone up their skin and attenuate the directly visible, unsightly physical changes that are linked to cutaneous aging.

The aging of the skin results from various alterations caused by factors that are both genetic and environmental. It manifests itself in particular by the loss of mechanical strength and viscoelastic and lifting properties of the dermis. The skin then has the tendency to stretch under the influence of its own weight, thus causing surface deformations, and the formation of wrinkles and unsightly folds. The epidermis also loses its thickness, and the cutaneous microrelief is modified.

To fight against this phenomenon, cosmetic active ingredients are therefore sought that make it possible both to lift and smooth the cutaneous microrelief, and to improve the viscoelastic properties of the skin at the same time.

To date, to meet their lifting needs, the formulators have had at their disposal two types of substances:
Synthetic and sticky polymers that are often difficult to formulate because they are only soluble in alcohol, and
Proteins.

Also, the purpose of this invention is another molecular means to eliminate the drawbacks of the prior art by proposing a process for obtaining an active ingredient with an immediate tensor effect that is effective, soluble and stable in water, of plant origin, and that limits the protein content as much as possible.

To this end, the invention has as its object a process for obtaining an active ingredient with an immediate anti-wrinkle effect and/or immediate tensor effect on the skin, characterized in that it consists in extracting and purifying polysaccharides of high molecular weight from oat bran and/or fibers and/or seeds and in solubilizing and stabilizing these polysaccharides in water.

The active ingredient according to the invention can be obtained from simple oat fibers and/or from oat bran, residue of the oat grounds obtained from the pericarp of seeds that, in addition to fibers, contains proteins, mineral salts, and vitamins, and/or from oat seeds.

Advantageously, the active ingredient that is obtained, polysaccharide-rich and of a high molecular weight, produces a sensation of stretched and toned skin and has an immediate tensor effect that is characterized by a smoothing of the cutaneous microrelief and an improvement in the mechanical properties of the skin, thus an immediate anti-wrinkle effect.

This invention is now described in detail by using non-limiting examples of compositions, as well as test results grouped in tables.

### I/Process for Obtaining the Active Ingredient According to the Invention

The process according to this invention comprises at least two essential stages:
A stage for solubilization of oat bran and/or fibers and/or seeds in a basic solution, and
A stage of successive or simultaneous enzymatic hydrolysis(es) of polysaccharides that are contained in the oat bran and/or fibers and/or seeds, so as to facilitate their solubilization without disrupting their molecular structure.

According to an embodiment of the invention, to facilitate the solubilization of polysaccharides, at least one adjuvant for solubilization in the basic solution, preferably a salt, a polyphosphate and/or an oxidizer, is added.

The concentration of alkaline agent of the basic solution for solubilization can be adjusted so that the physical properties of the polysaccharides are not altered by simple sugars during hydrolysis.

Preferably, the process according to this invention also comprises a deproteinization stage.

According to a preferred embodiment, the process according to the invention comprises the series of the following stages:
Solubilization of an oat bran and/or fibers and/or seeds at a rate of 30 g/l to 300 g/l, more particularly from 50 g/l to 150 g/l, in a basic solution,
Successive or simultaneous enzymatic hydrolysis(es) of polysaccharides,
Inactivation by heat or chemical treatment to block the enzymatic reactions,
Separation of the soluble and insoluble phases by filtration, decanting, and/or centrifuging,
Successive concentrations,
Deproteinization by precipitation or selective adsorption,
Purification of the active fraction that contains the polysaccharides of high molecular weight by ultrafiltration, and
Sterilizing filtration.

Advantageously, the process according to the invention allows the preservation of native polysaccharides that are derived from oat bran and/or fibers and/or seeds, while facilitating the industrial feasibility of the active ingredient.

II/Characterization of the Active Ingredient that is Obtained According to the Invention from Oats

### II.1/Dry Material

The level of dry material is measured by running a sample with a given initial weight through the oven at 105° C. until a constant weight is obtained.

The level of dry material is between 20 and 200 g/l, more particularly between 60 and 110 g/l.

### II.2/Measurement of pH

The pH that is measured by the potentiometric method at ambient temperature leads to values of between 4 and 8, more particularly between 5 and 6.

### II.3/Determination of the Content of Total Sugars

The method of DUBOIS (DUBOIS, M. & al. [1956], Analytical Chemistry, 28, No. 3, pp. 350-356) is used.

In the presence of concentrated sulfuric acid and phenol, the reducing sugars provide a yellow-orangey compound.

Starting from a standard range, it is possible to determine the level of total sugars of a sample.

The level of total sugars of the active ingredient according to this invention is 19 to 190 μl, preferably 57 to 105 μl.

The ratio of the total sugars to the level of dry material for the active ingredient according to this invention is greater than 50%, preferably greater than 80%.

### II.4/Mean Polymerization Degree of Polysaccharides

The mean polymerization degree of polysaccharides is determined by the ratio of the level of total sugars to the level of reducing sugars.

The metering of reducing sugars is carried out as follows:
The active ingredient to be metered is brought into the presence of a solution of 4-hydroxybenzoic hydrazide in 0.5 M hydrogen chloride and a 0.5 M soda solution,
A standard range is produced with glucose, and
The absorbance is measured at 410 nm to determine the content of reducing sugars of the active ingredient relative to the glucose range.

The mean polymerization degree of the polysaccharides of the active ingredient according to this invention is greater than 40, preferably greater than 60.

### II.5/Polysaccharide Size

The distribution by size of the polysaccharides that are obtained by the implementation of the process according to the invention is carried out by studying the chromatograms.

The polysaccharides that are obtained by the implementation of the process according to the invention are polysaccharides of high molecular weight. They have a polysaccharide size of between 30 and 2,000 kDa. Preferably, 50% of the polysaccharide fraction has a size of between 70 and 700 kDa.

III/Evaluation of the Effect of the Active Ingredient that is Obtained According to the Invention from Oats

III-1/Evaluation of the Tensor Effect by Cutometer

This study has as its objective to evaluate the tensor effect of an active ingredient that is obtained according to the invention from oat bran.

The study is performed on volunteers using a Cutometer.

A Cutometer is a device that is equipped with a probe that is applied to the skin in which a constant depression is maintained. The depth of penetration of the skin in the probe is measured under the intake effect.

When subjected to these depressions, the skin becomes tired more or less quickly and the response times as well as the measured amplitudes make it possible to determine the parameters, in particular:
An elastic component, Ue, which corresponds to an instantaneous deformation, and
Uf, which corresponds to the extensibility.

If Ue decreases, the skin is less flexible and therefore more stretched.

If Uf decreases, the skin is less extensible, and therefore also more stretched.

The operating protocol is as follows:
A zone is identified on the volunteers' forearms, and a first series of measurements is taken with the Cutometer,
The active ingredient that is derived from oat bran that is obtained according to the invention at 4% in emulsion or a placebo is applied to the identified zone, and
Two hours after the application, a new series of measurements is taken on the identified zone.

As reference results, the BSA (bovine serum albumin) metered at 4% is used.

The results that are obtained for the active ingredient that is derived from oat bran according to the invention are expressed relative to the placebo in the table below:

| | Cosmetic Effectiveness/Placebo | |
|---|---|---|
| | (ΔUf) | (ΔUe) |
| 4% BSA | −5.0% | −7.1% |
| Active Ingredient According to | −8.4% | −9.9% |
| the Invention | | |

It is noted that the active ingredient according to the invention reduces the elastic component and the extensibility of the skin: it has an immediate tensor effect on the skin.

III-2/Evaluation of the Tensor Effect in Sensory Analysis

The objective of this study is to quantify in vivo the tensor effectiveness of an active ingredient according to the invention, obtained from oat bran, formulated at 10% of counter-placebo gel.

The sensory evaluation test consists in having a panel of experts make a blind evaluation of the tightening and non-sticky sensation. The study is performed on 15 healthy volunteers at the level of the eye and the crow's-feet.

The operating protocol is as follows:
At T minus 5 minutes, the volunteers remove make-up from the selected eye and crow's-feet,
At T0, 80 μl of a gel that is to be tested is applied: placebo gel, gel that contains 10% of the active ingredient according to the invention that is derived from oat bran, gel that contains 5% BSA, gel that contains 10% BSA, and gel that contains 20% BSA, and
At T 3 minutes, T 5 minutes, and T 10 minutes, the tightening sensation is evaluated on a scale of 0 to 10, using a cursor.

The analysis of the scales is carried out by totaling the scores over three cycles.

The various gels are tested randomly over several days (one gel per day).

The results that are obtained are presented in the table below:

| | Total of the Scores over 3 |
|---|---|
| | Cycles |
| Placebo | 4.1 |
| Active Ingredient According to the | 13.6 |
| Invention at 10% | |
| 5% BSA | 8.2 |
| 10% BSA | 12.9 |
| 20% BSA | 14.6 |

It is noted that after a single application, the experts identify the active ingredient according to the invention as tightening and non-sticky, and score it at an effectiveness of 13.6, which is higher than that of BSA metered at 10%.

III-3/Evaluation of the Immediate Anti-Wrinkle Effect

The object of this study is to quantify the immediate anti-wrinkle effectiveness of an active ingredient according to the invention, obtained from oat seeds, formulated at 4% in counter-placebo emulsion.

The study is performed on healthy female volunteers.

Anti-wrinkle effectiveness is measured by means of silicone imprints made in the crow's-feet of volunteers.

The analysis of these imprints using a profilometer equipped with an image analyzer makes it possible to obtain three parameters: the number of wrinkles, the total wrinkled surface area, and the total length of the wrinkles.

The study is performed according to the following protocol.

At T0, two symmetrical cutaneous zones are identified at the crow's-feet—one intended to be treated by placebo, the other by the active ingredient—and imprints are made of these two zones.

After the imprints are made, the placebo and the active ingredient according to the invention, derived from oat seeds and formulated at 4%, are applied to the defined zones.

At T 2 hours, the imprints are made on the two zones that are being studied.

The results that are obtained for the active ingredient according to the invention, derived from the oat seeds, are expressed in the table below by percentage relative to those obtained for the placebo:

| | Variation/Placebo (%) |
|---|---|
| Number of Wrinkles | −11.5 |
| Total Wrinkled Surface Area | −17.4 |
| Total Length | −13.9 |

It is noted that after two hours, in comparison to the placebo, the active ingredient according to the invention that is formulated at 4% reduces the number of wrinkles, the total wrinkled surface area, and the total length of the wrinkles at the same time. It therefore has an immediate anti-wrinkle effect.

III-4/Evaluation of the Tensor Effect in Sensory Analysis

The objective of this study is to quantify in vivo the tensor effectiveness of the active ingredient according to the invention, obtained from oat seeds, formulated at 4% of counter-placebo gel.

The sensory evaluation test consists in having a panel of experts make a blind evaluation of the tightening and non-sticky sensation, formed with this tightening sensation. The study is performed on 15 healthy volunteers at the level of the eye and the crow's-feet.

The operating protocol is as follows:
At T minus 5 minutes, the volunteers remove make-up from the selected eye and crow's-feet,
At T0, 80 μl of a gel that is to be tested is applied: a placebo gel or a gel that contains 4% of the active ingredient according to the invention that is derived from oat seeds, and
At T 3 minutes, T 5 minutes, and T 10 minutes, the tensor sensation is evaluated on a score scale that ranges from 0 to 10, using a cursor.

The results that are obtained, corresponding to the mean of the scores with three cycles, are presented in the table below:

| | Mean Score |
|---|---|
| Placebo | 2.1 |
| Active Ingredient According to the | 4.4 |
| Invention at 4% | |

It is noted that after a single application, the experts identify the active ingredient according to the invention as tightening and non-sticky, and score it at an effectiveness of 4.4.

### IV/Cosmetic Composition Including the Active Ingredient According to the Invention:

This invention also covers the cosmetic compositions including the active ingredient according to this invention in various galenical forms, in particular gel, solution, emulsion, cream, . . . .

It is then advisable to analyze the stability of the galenical forms, including the active ingredient according to the invention, in proportions of between 1 and 5%.

The stability is characterized by an absence of precipitation of the active ingredient, an absence of creaming, and an absence of phase shift.

It is possible to cite formulations that have shown a physical stability that includes 5% of active ingredient according to the invention.

### Clear Gel:

Carbopol: 0.5% with triethanolamine: sufficient quantity for pH=6.5
Preservative: 0.7%
Active ingredient: 5.0%
Water: 93.8%

### Opaque Gel:

Sepigel 305: 2.0%
Preservative: 0.7%
Active ingredient: 5.0%
Water: 92.3%

### Emulsified Gel:

Montanov 202: 3.0%
Isopropyl palmitate: 12.0%
Preservative: 0.7%
Viscolam AT 64: 2.0%
Active ingredient: 5.0%
Water: 77.3%

### Non-Ionic Emulsion:

Montanov 202: 3.0%
Simulsol 165: 2.0%
Isopropyl palmitate: 20.0%
Preservative: 0.7%
Active ingredient: 5.0%
Water: 69.3%

### Anionic Emulsion:

Stearic acid: 7.0%
Triethanolamine: 3.5%
Isopropyl palmitate: 20.0%
Preservative: 0.7%
Active ingredient: 5.0%
Water: 63.8%

### Cationic Emulsion:

Quaternium-82: 5.0%
Cetyl alcohol: 2.0%
Cetearyl alcohol: 1%
PEG100 stearate: 1%
Isopropyl palmitate: 15.0%
Preservative: 0.7%
Active ingredient: 5.0%
Water: 70.3%

In addition, tests have shown the compatibility of the active ingredient with the raw material used in cosmetics.

1. Process for obtaining an active ingredient with an immediate anti-wrinkle effect and/or immediate tensor effect on the skin, characterized in that it consists in extracting and purifying polysaccharides of high molecular weight from oat bran and/or fibers and/or seeds and in solubilizing and stabilizing these polysaccharides in water.

2. Production process according to claim 1, wherein it comprises at least the following two stages:
Solubilization of oat bran and/or fibers and/or seeds in a basic solution, and
Successive or simultaneous enzymatic hydrolysis(es) of polysaccharides that are contained in the oat bran and/or fibers and/or seeds.

3. Production process according to claim 2, wherein it comprises a deproteinization stage.

4. Production process according to claim 1, wherein it comprises the series of the following stages:
Solubilization of oat bran and/or fibers and/or seeds in a basic solution, at a rate of 30 g/l to 300 g/l,
Successive or simultaneous enzymatic hydrolysis(es) of polysaccharides that are contained in the oat bran and/or fibers and/or seeds,
Inactivation by heat or chemical treatment to block the enzymatic reactions,
Separation of soluble and insoluble phases by filtration, decanting, and/or centrifuging,
Successive concentration(s),
Deproteinization by precipitation or selective adsorption,
Purification of the active fraction that contains the polysaccharides of high molecular weight by ultrafiltration, and
Sterilizing filtration.

5. Production process according to claim 4, wherein the solubilization of oat bran and/or fibers and/or seeds in a basic solution is done at a rate of 50 g/l to 150 g/l.

6. Production process according to claim 2, wherein at least one solubilization adjuvant is added in the basic solution.

7. Production process according to claim 6, wherein the solubilization adjuvant is a salt, a polyphosphate and/or an oxidizer.

8. Active ingredient that is obtained by the implementation of the process according to claim 1, characterized by the following parameters:
Level of dry material of between 20 and 200 g/l,
pH of between 4 and 8,
Content of total sugars of between 19 and 190 g/l,
Presence of polysaccharides of high molecular weight, which present a degree of polymerization of more than 40.

9. Active ingredient according to claim 8, characterized by the following parameters:
Level of dry material of between 60 and 110 g/l,
pH of between 5 and 6,
Content of total sugars of between 57 and 105 g/l,
Presence of polysaccharides of high molecular weight, which have a degree of polymerization of more than 60.

10. Composition that includes the active ingredient according to claim 8, wherein it consists of a clear gel, an opaque gel, an emulsified gel, a non-ionic emulsion, an anionic emulsion, or a cationic emulsion.

11. A cosmetic composition, comprising the polysaccharides of high molecular weight that are obtained from oat bran and/or fibers and/or seeds and by implementation of the process according to claim 1, wherein the cosmetic composition provides an immediate cutaneous tensor effect.

12. A cosmetic composition comprising polysaccharides of high molecular weight that are obtained from oat bran and/or fibers and/or seeds and by implementation of the process according to claim 1, wherein the cosmetic composition produces tight and toned skin.

13. A cosmetic composition comprising polysaccharides of high molecular weight that are obtained from oat bran and/or fibers and/or seeds and by implementation of the process according to claim 1, wherein the cosmetic composition has an immediate anti-wrinkle effect.

14. A method of smoothing of cutaneous microrelief and improving the mechanical properties of skin comprising administering to a subject in need thereof an effective amount of polysaccharides of high molecular weight that are obtained from the oat bran and/or fibers and/or seeds.
